(19) United States
(12) Patent Application Publication
(10) Pub. No.: US 2005/0137509 A1
(43) Pub. Date: Jun. 23, 2005

(54) Back support

(76) Inventors: Karine Ishkhanova, Ontario (CA); Mikhail Ishkhanov, Ontario (CA)

Correspondence Address: WATTS, HOFFMANN CO., L.P.A., Ste. 1750, 1100 Superior Ave., Cleveland, OH, 44114, US

(21) Appl. No.: 11/010,884

(22) Filed: Dec. 13, 2004

(30) Foreign Application Priority Data
Dec. 22, 2003 (CA) 2,451,714

(51) Int. Cl. A61F 5/00

(52) U.S. Cl. 602/19

(57) ABSTRACT

A waistband with sculptured soft layer for providing back support as needed by the user. It is intended to be employed when the user is lying on a back or is in a sitting position. The waistband includes a base portion having either two cooperating ends to surround a user's lower back or forming an integral part of the garment, and the sculptured portion having inner and outer surfaces where the inner surface positioned against the base portion of the waistband and the outer surface positioned against the body of the user. The inner surface of the sculptured portion forms a flat plane parallel to the base portion of the waistband and the outer surface forms a curved shape conforming to the contours of the user's body.

### FIELD OF THE INVENTION

This invention relates to a new back support waistband with self-adjustable sculptured layer.

### BACKGROUND OF THE INVENTION

Known methods for providing a back support while resting on a bed include using supports that are disconnected from the body like a rolled up towel, a pillow, etc. Generally these supports have a major disadvantage of being difficult to sleep with due to shifting during the night and sometimes may even lead to an increased back pain because of the formation of unbalanced pressure points. Another option for a back support involves use of a specialized orthopedic mattresses (e.g. Canadian Patent Application # CA 2368269 and Patent Application # CA 2239121). While this alternative presents a valid solution it comes at a high cost to the user and is not easily interchangeable. It also has a drawback of being a stationary solution and as such can not be taken along by a user during the day or while traveling on trips.

Consequently a need exists for an inexpensive, portable and simple to use back support, which will fit a broad range of users and will be easily interchangeable if a need arises.

### SUMMARY OF THE INVENTION

The present disclosure concerns a waistband with a sculptured soft layer for providing back support as needed by a user. It is intended to be employed when a user is lying on his or her back or is in a sitting position. One embodiment includes a base portion and a sculptured portion. The base portion either has two cooperating ends or forms an integral part of the garment or self-adheres, so that the waistband can be positioned against the user's lower back. The sculptured portion has inner and outer surfaces where the inner surface is positioned against the base portion of the waistband and the outer surface faces the user's body. In one embodiment, the inner surface of the sculptured portion forms a flat plane parallel to the base portion of the waistband and the outer surface forms a curved shape conforming to the contours of the user's body. The operative volume of this sculptured portion is determined by the space formed as an intersection of flat plane of the inner surface and curved shape of the outer surface.

The base portion and the sculptured portion are designed to be related to each other in one of the following ways:
releasably connected by means of cohesive-adhesive material
releasably connected by way of inserting the sculptured portion inside the base portion
non-releasbly connected by such way of manufacturing that merges the base and sculptured portions into a single unit.

Releasable connection between the base and sculptured portions offers an advantage of using interchangeable customized sculptured portions, while the non-releasable connection offers an advantage of simpler to maintain, especially when it's outer surface is made of such easy to maintain and hygienically sound materials as silicon, neoprene blend etc.

The base portion of the waistband is preferably made of a stretchable material to be able to adjust to the size of the user's waist and back.

The sculptured portion of the waistband is either made of or contains a soft fill such as foam, silicon, neoprene, polyester, nylon, etc. or mixtures of thereof providing that it's outer surface will conform to a contour specifics of a user's body. The sculptured portion should also be resilient in order to hold the shape and thus to support a natural sleeping posture. The sculptured portion may contain a core part made of rigid material e.g. rubber, dense foam, dense silicon etc. to provide additional support, particularly important for heavier users because their middle body creates extensive pressure on their spine while in a lying position. The outer surface of the sculptured portion or the waistband as a whole may contain a self-adhesive layer allowing it to adhere directly to the user back. This option offers an advantage of more precise positioning against the user's back, but at the same time should not be the choice for people with the sensitive skin.

The waistband is designed to allow it to be worn both under the clothes or over the clothes, as well as the waistband or it's sculptured portion may comprise an integral part of the garment e.g. pajama top, nightgown, nightshirt, T-shirt, vest, etc.

The waistband support is designed to maintain the back of a user in a naturally aligned position, when a user is lying on a back. The waistband's sculptured layer conforms to a user's movement while asleep to provide adequate back support on a change of position from the side to the back and vice versa. This helps to reduce stiffness, discomfort, muscle strain and aching.

The waistband's structure and design characteristics also make it very useful for helping to maintain natural posture while in a sitting position. Unfortunately not every company provides their employees with the comfortable, adjustable and health oriented sitting chairs. This waistband may become a solution helping employees of such professions as researches, clerks, programmers, operators, accountants etc. to prevent back stiffness, discomfort, muscle strain and aching caused by the long work hours in a sitting position.

### BRIEF DESCRIPTION OF THE DRAWINGS

FIG. 1 is a front 3-Dimensional view of the waistband of the present invention containing two cooperating ends;

FIG. 2 is a cross-sectional view of the waistband of FIG. 1 as seen from the plane 2-2 of FIG. 1;

FIG. 3 is a back view of the waistband being worn by a person lying on a side;

FIG. 4 is a side view of the waistband supporting back of a person lying on a back;

FIG. 5 is a side view of the waistband used to support natural posture of a person sitting on a chair;

FIG. 6 is a front 3-Dimensional view of the waistband that forms an integral part of a garment;

FIG. 7 is a flat vertical cross-sectional view of the waistband being an integral part of a garment;

FIG. 8 is a side view of a person wearing the garment with an integral waistband used to support natural posture while sitting on a chair;

FIG. 9 is a back view of a person wearing the night garment with an integral waistband used while in a side lying position; and

FIG. 10 is a side view of a person wearing the night garment with an integral waistband supporting back while in a back lying position.

### DESCRIPTION OF THE PREFERRED EMBODIMENTS

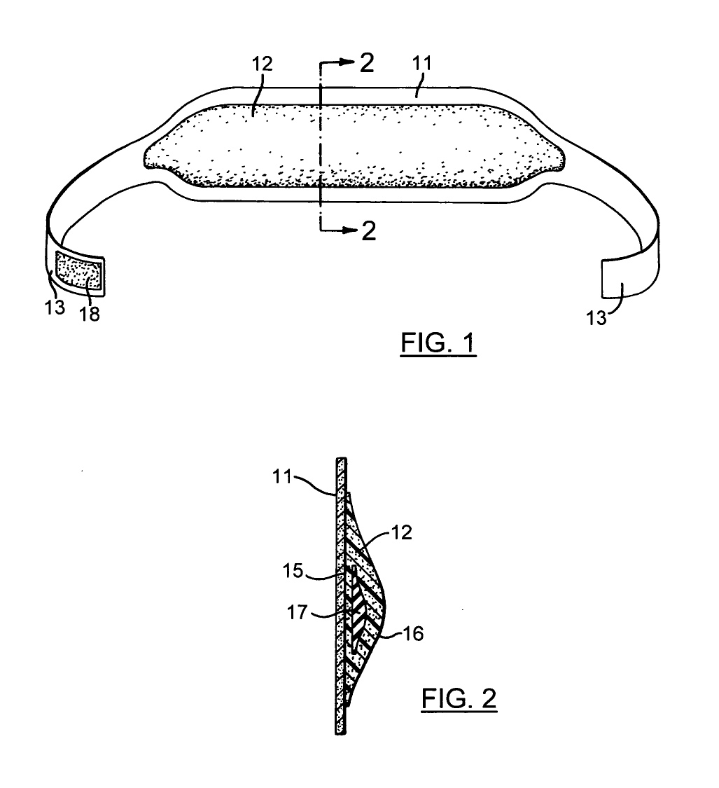

Particularly advantageous embodiments of the invention are illustrated in FIGS. 1 through 10.

FIGS. 1 through 5 illustrate a waistband having
a base portion, indicated generally at 11, preferably fabricated from a stretchable material and having two cooperating ends, generally indicated at 13; and
a sculptured portion, indicated generally at 12, preferably fabricated from a soft molded material like foam, silicon, neoprene, polyester, nylon etc. or mixtures of thereof.

As depicted in the FIG. 2 cross-sectional view of the waistband of FIG. 1, the volume of the sculptured portion 12 is defined by an inner surface 15 parallel to the flat plane of the base portion 11 and by an outer surface 16 of curved shape conformed to the contours of a human body.

A rigid core 17 particularly visible in FIG. 2 creates additional support and preferably is manufactured from rubber, dense foam, etc.

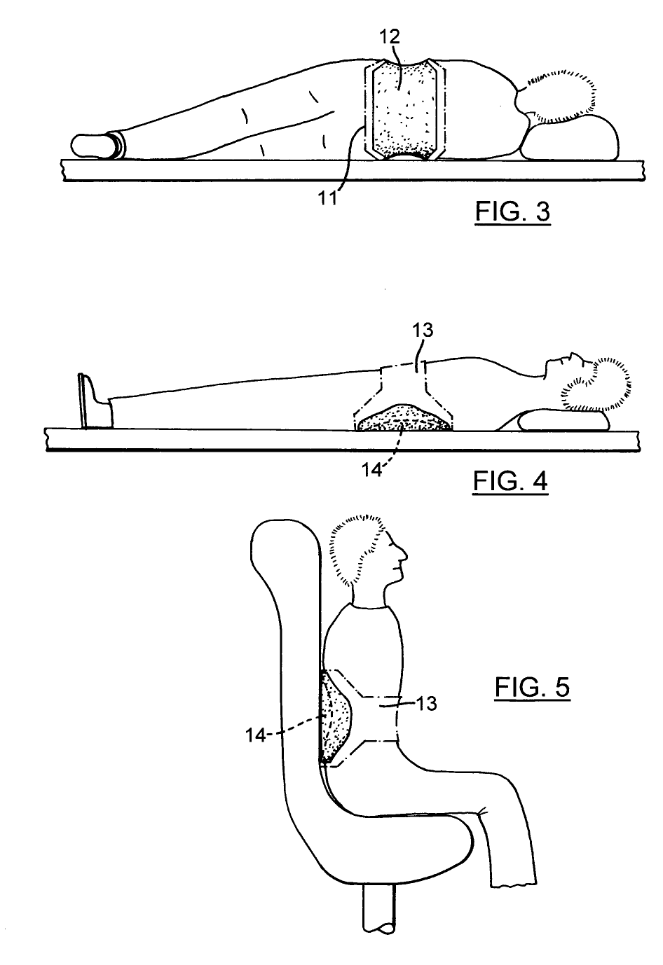

As illustrated in FIG. 3 the waistband is used by a person lying on a side with his back facing viewer. The waistband ends 13 are secured together by means of a Velcro patch 18 near an endmost portion of the waistband. The FIG. 3 depiction demonstrates that the sculptured portion 12 turns as the user turns and thus does not interfere with a sleeping surface.

As illustrated in FIG. 4 the waistband is used by a person lying on a back when his/her back is supported by the part 14 of a sculptured portion of the waistband.

As illustrated in FIG. 5 the waistband is used by a person sitting on a chair when his/her natural position is supported with the help of the part 14 of a sculptured portion of the waistband.

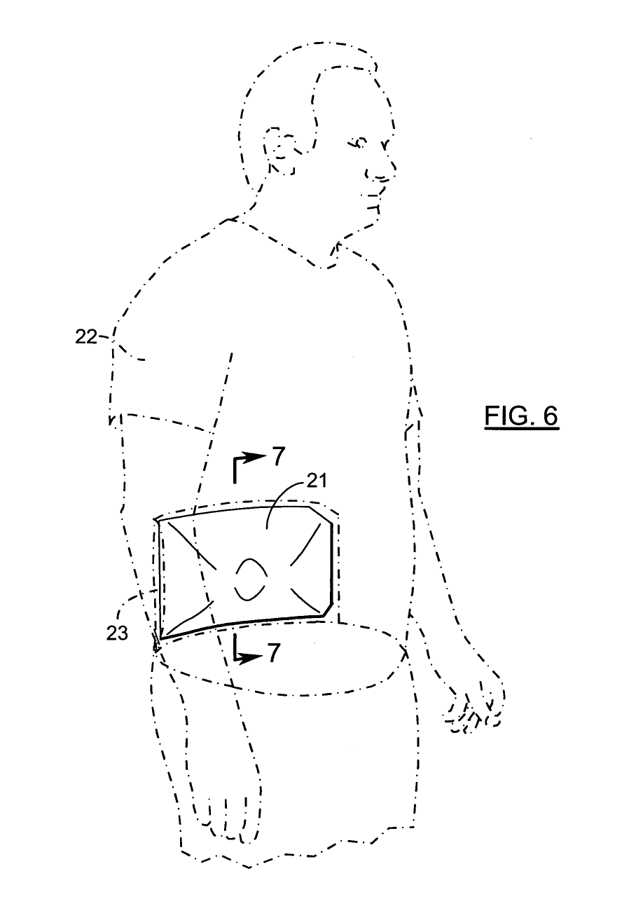

FIGS. 6 through 10 illustrate a second embodiment of the waistband, when it forms an integral part of the garment.

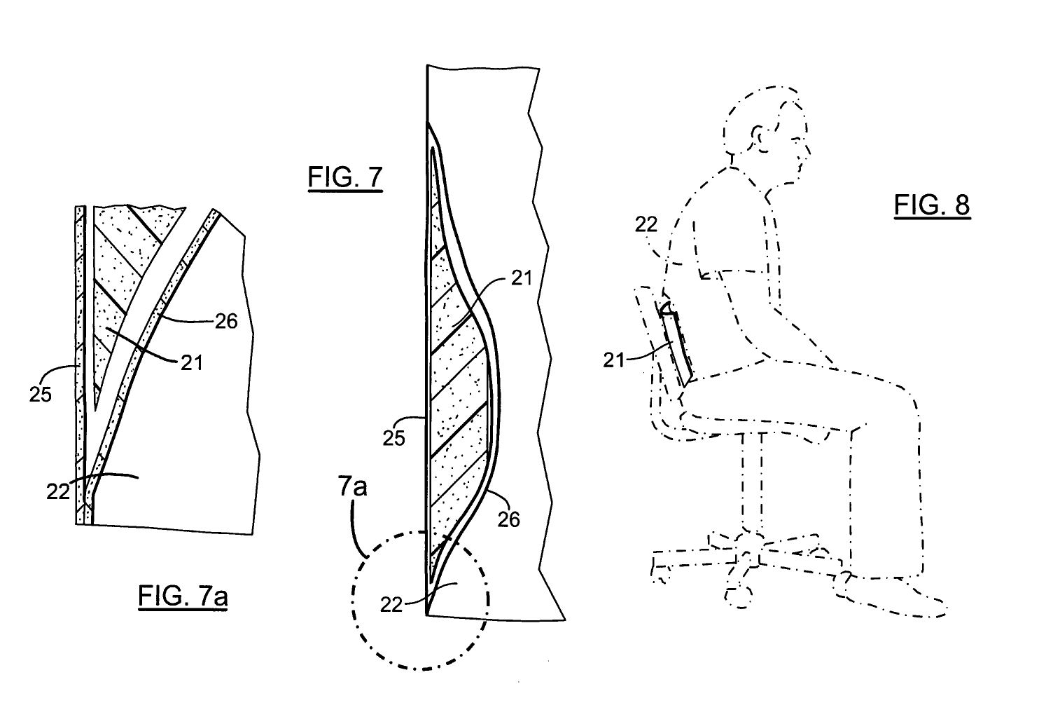

As illustrated in FIG. 6 the sculptured part 21 is inserted into base portion with the opening 23 sewn in as an integral part of the garment 22. Contour lines in FIG. 6 illustrate that the sculptured part 21 is thinner around its outer periphery and thicker at its center. The section view of FIG. 2 illustrates that the sculptured part 21 can increase in thickness in a generally symmetric uniform manner with respect to a centerline of the part 21. FIG. 7 illustrates that the thickness can increase asymmetrically with the increase occurring more gradually on an upper portion that along a lower portion.

As depicted in FIG. 7 representing cross-sectional view of the garment with the build in waistband, sculptured part 21 is located in the inner part of the garment thus creating a more discrete and pleasant outlook. In FIG. 7A inner 25 and outer 26 fabric layers are seen in a region where they are sewn together to form a pocket or envelope for supporting the sculptured part 21. The sculptured part 21 can either be a permanent part of the garment or, if the sewing only borders a part of the envelope or pocket to leaving an access opening, the part can be removed to allow interchangeability.

As illustrated in FIG. 8 the garment 22 with the build in waistband is used by a person sitting on a chair when his/her natural position is supported with the help of the sculptured portion 21 of the waistband.

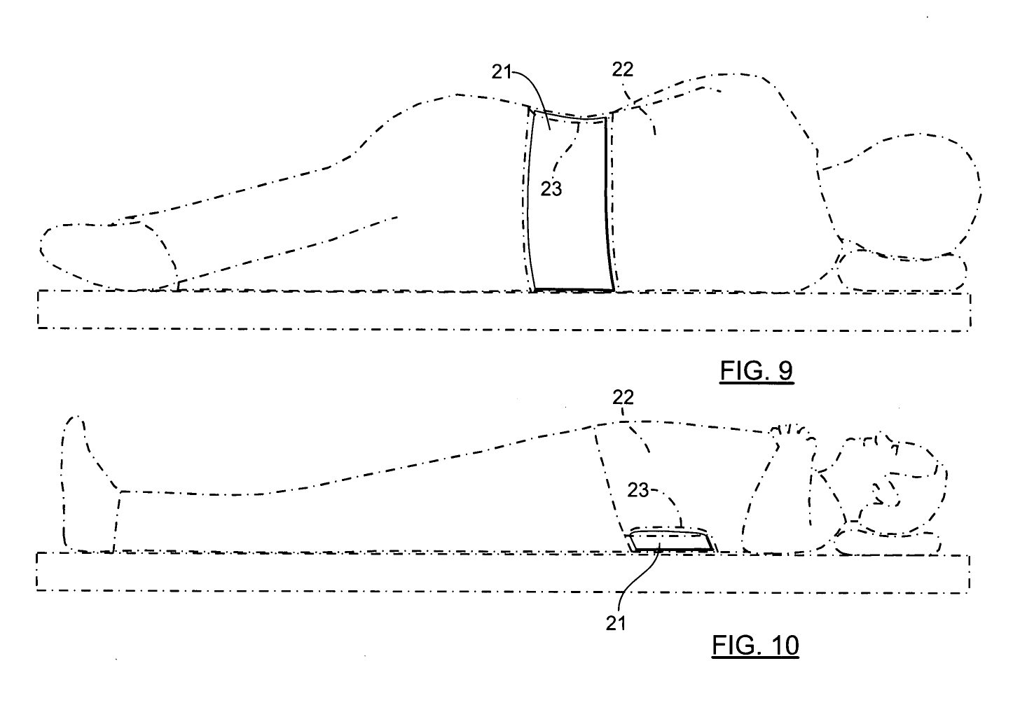

As illustrated in FIG. 9 the garment 22 is worn by a person lying on a side with his back facing viewer which demonstrates that the sculptured portion 21 turns together with a user thus not interfering with the sleeping surface.

As illustrated in FIG. 10 the garment 22 is worn by a person lying on a back when his/her back is supported by the sculptured portion 21 inserted into the base portion with the opening 23.

Although the invention has been described in connection with a preferred embodiments, it should be understood that various modifications, additions and alterations may be made to the invention by one skilled in the art without departing from the spirit and scope of the invention as defined in the appended claims.

1. A body support comprising:
a base portion;
a sculptured portion having inner and outer surfaces where the inner surface positioned against and is supported by the base portion and the outer surface is positioned against the user's body.

2. The support of claim 1 wherein said base portion and said sculptured portion are releasably connected by means of cohesive-adhesive material.

3. The support of claim 1 wherein said base portion and said sculptured portion are releasably connected by way of inserting the sculptured portion into the provided opening in the base portion.

4. The support of claim 3 wherein said base portion comprises an envelope structure having at least one opening to allow insertion of the sculptured portion into it.

5. The support of claim 1 wherein said base portion and said sculptured portion are non-releasably connected by way of manufacturing that merges the base and sculptured portions into a single unit.

6. The support of claim 1 wherein said base portion is partially or entirely made of stretchable material.

7. The support of claim 1 wherein said base portion has two cooperating ends to surround a user's lower back.

8. The support of claim 7 wherein cooperating ends of said base portion are releaseably connected by means of cooperating fasteners or cohesive-adhesive materials.

9. The support of claim 7 wherein cooperating ends of said base portion are non-relesably connected, so that the waistband is put on by way of user slipping into it through a person's legs or head.

10. The support of claim 1 wherein said base portion is partially or entirely made of non-stretchable material.

11. The support of claim 1 wherein said base portion forms an integral part of the garments such as pajamas, nightgowns, nightshirts, T-shirts, vests.

12. The support of claim 1 wherein the inner surface of said sculptured portion forms a flat plane parallel to the base portion.

13. The support of claim 1 wherein the outer surface of said sculptured portion forms a curved shape conformed to the contours of the user's body.

14. The support of claim 1 wherein the operative volume of said sculptured portion is determined by the space formed as an intersection of flat plane of the inner surface and curved shape of the outer surface.

15. The support of claim 1 wherein the said sculptured portion is made partially or entirely of such materials as silicon, foam, neoprene, nylon, down, feathers, gel, mixtures of thereof.

16. The support of claim 1 wherein the said sculptured portion contains a core part made of rigid resilient material such as rubber, dense foam, dense silicon.

17. The support of claim 1 comprising a kit wherein the said sculptured portion is distributed separately from the base portion to serve as an interchangeable part used in conjunction with the base portion.

18. The support of claim 1 comprising a kit wherein the said base portion is distributed separately from the sculptured portion to serve as an interchangeable part used in conjunction with the sculptured portion.

19. The support of claim 1 wherein the said sculptured portion is used as an integral part of such garments as pajamas, nightgowns, nightshirts, T-shirts, vests.

20. The support of claim 1 wherein the outer surface positioned against the user body contains a self-adhesive layer allowing the surface to adhere directly to a user's body.

21. The support of claim 1 wherein the sculptured portion has a self-adhesive layer on the outer surface positioned against the user body thereby allowing the outer surface to adhere directly to a user's body.

22. The support of claim 1 wherein the said waistband is used as an integral part of such garments as pajamas, nightgowns, nightshirts, T-shirts, vests.

23. A garment such as pajamas, nightgowns, nightshirts, T-shirts, shirts or vests comprising
a base portion;
a sculptured portion having inner and outer surfaces where the inner surface positioned against and is supported by the base portion and the outer surface is positioned against the user's body.

24. The garment of claim 23 containing as an integral part the sculptured portion.

25. A garment of claim 23 containing as an integral part the base portion and an interchangeable sculptured portion.
